(19) United States
(12) Patent Application Publication
(10) Pub. No.: US 2003/0206862 A1
(43) Pub. Date: Nov. 6, 2003

(54) Contrast medium based on polyoxyethylene-660-12-hydroxystearate and anionic phospholipids

(76) Inventor: Thomas Gieselmann, Kastellaun (DE)

Correspondence Address: Pauley Petersen Kinne & Erickson, 2800 W. Higgins Road, Suite 365, Hoffman Estates, IL, 60195, US

(21) Appl. No.: 10/152,151

(22) Filed: May 21, 2002

Related U.S. Application Data
(63) Continuation-in-part of application No. 09/601,759, filed on Aug. 7, 2000, now abandoned.
Application No. 09/601,759 is a 371 of international application No. PCT/EP99/00649, filed on Feb. 2, 1999.

(30) Foreign Application Priority Data
Feb. 7, 1998 (DE) 198 05 012.7

(51) Int. Cl. A61K 49/00

(52) U.S. Cl. 424/9.51

(57) ABSTRACT

An IV contrast medium containing a foamed preparation based on an aqueous solution of polyoxyethylene-660-12-hydroxystearate and an anionic phospholipid, and a method for producing the contrast medium, and to its use as a diagnostic reagent in imaging procedures.

### BACKGROUND OF THE INVENTION

1. Field of the Invention

This invention relates to a contrast medium which can be intravenously (IV) administered for use as a diagnostic agent during image-producing methods, and to a method for producing the contrast medium.

2. Description of Related Art

Ultrasound diagnosis has found a very wide range of use in medicine because of its complication-free and simple application. The principle of ultrasound diagnosis is based on the fact that sound waves are differently reflected by organs and their outlines. These echo signals are made visible by electronic amplification.

The representation of blood flow in vessels and inner organs is not possible in a normal two-dimensional image (B mode). Blood and other liquids only provide an ultrasound contrast if there are differences in density in relation to the surroundings.

In medicine, air or other gases are respectively used as contrast media in the ultrasound range, because the impedance shift between the gas and the surrounding blood is considerably larger than the one between liquids or solids and blood.

In connection with ultrasound examination of the heart or vessels, it is possible to use the weak reflections from red cells by making use of a Doppler phenomenon. It is thus possible to make the blood flow visible without the addition of a contrast medium.

However, in order to be able to detect the blood flow in deeper-lying vessels or respectively to detect very low flow velocities, the admixture of small gas bubbles into the bloodstream is advantageous, because the stronger reflection thus caused makes a better diagnosis possible.

The respirable echo contrast media, known from literature, function on the basis of foreign gases. So far there has been no information as to whether these contrast media on the basis of air would also be respirable, see, for example, Echocardiography: A Jrnl. of CV Ultrasound & Allied Tech., vol. 14, no.5, 1997, pp. 441 to 446, by Thomas R. Potter, M.D., Feng Xie, M.D. and Shouping Li, M.D. “Differences in Myocardial Contrast Produced with Transient Response Imaging When Using Intravenous Microbubbles Containing Gases of Different Molecular Weight”, or “Drugs of the Future 1995, 20 (12): 1224 to 1227 “Levovist^{(R)}” by Dr. Thomas Fritzsch and Dr. med. Reinhard Schlief, or Echocardiography: A Jrnl. of CV Ultrasound & Allied Tech., vol. 14, no. 4, 1997, pp. 337 to 343, by Sameh Mobarak, M.D., Marc Kates, D.O., Maria Meza, M.D., Carlos Moreno, Susan Revall, Wayne Barbee, Ph.D. “Identification of Perfusion Abnormalities Using FS069, a Novel Contrast Agent, in Conscious Dogs”, or Acta Radiologica 38 (1997), Supplement 412, 101 to 112 by J. -M. Correas, O. Hélénon, L. Pourcelot and J. -F. Moreau “Ultrasound Contrast Agents”.

Several methods are known from such literature for the production and subsequent stabilization of air bubbles or respectively gas bubbles. Microbubbles can be created by intense shaking, or respectively stirring of solutions, such as common salt or dye solutions, or of previously drawn blood. Although a contrast in the ultrasound is thus achieved, it is connected with one disadvantage of poor reproducibility and greatly differing size of the small gas bubbles. These disadvantages are partially removed by improved methods.

For increasing the contrast, preparations are used in ultrasound diagnosis which contain extremely finely distributed small gas bubbles, the same as agitated X-ray contrast media, for example. Galactose particles are described in the literature, on which air is adsorbed and which are released as soon as they come into contact with a suitable solvent.

A means for the use in ultrasound diagnosis containing microparticles, which are coated with primarily lipophilic materials and therefore prevent a rapid dissolution of the galactose, is described in German Patent Reference DE 44 06 474 A1. These microparticles are filled into a container, are coated with gaseous halogenated hydrocarbon and are suspended in water before use. This is a suspension of microparticles in water or in an aqueous solution.

A contrast medium is described in PCT International Application WO 93/13808, wherein microparticles, for example galactose particles, are coated with an emulsifying agent. The particles are suspended in an aqueous solution immediately before use. These are also particles in a suspension.

Stabilized preparations as ultrasound contrast agents are described in PCT International Application WO 96/26746, which contain a hydrophilic material, for example hydroxyethyl starch, in combination with a neutral phospholipid (phosphatidyl choline) and a further emulsifying agent. The mixture is applied after the addition of an aqueous solvent and a suitable gas. No negatively charged (anionic) phospholipid is used, and liposomes (particles) are present instead of a solution. The liposomes, or surface-active viscous solutions, or a solution in accordance with German Patent Reference DE 43 28 642 A1 is sonocated with physiologically compatible gas and is stored deep frozen.

A foamed and subsequently denatured protein is described, as well as a substance respectively containing a surfactant or a surfactant mixture and a viscosity-increasing substance.

Aqueous preparations for use as echo contrast media are known from German Patent Reference DE 196 26 530 A1, German Patent Reference DE 43 28 642 A1, European Patent Reference EP 0 494 615 A1, and PCT International Application WO 92/11873, which are based on a mixture of polyoxyethylene-poly-oxypropylene-polymers with negatively charged phospholipids, which are in a clear solution.

In connection with the presently available contrast media, mainly two factors play an overriding role with respect to possible risks: the size and number of the small gas bubbles and of the solid particles.

### SUMMARY OF THE INVENTION

One object of this invention is to create an ultrasound contrast medium having the following required properties:

1. Respirability;

2. Minimization of the risk of an embolism

small gas bubbles (size and number)

solid particles (size and number);

4. Reproducibility;

5. Sterile preparation free of pyrogenes;

6. Simple manufacture at reasonable cost;

7. Problem-free storage;

8. Sufficiently long shelf life; and/or

9. Economical application.

In accordance with the invention, this object is attained by an IV contrast medium and the method for producing it in accordance with the following specification and the claims. In accordance with this invention, a preparation containing an aqueous solution of polyoxyethylene-660-12-hydroxystereate and an anionic phospholipid is used as contrast medium for intravenous (IV) administration.

Thus this invention proposes a clear, aqueous solution of a mixture of two surfactant substances for attaining the intended object, which also possibly contains substances for isotonization, and into which a physiologically compatible gas is introduced prior to use.

A preparation in accordance with this invention is further developed into a contrast medium by the incorporation of a physiologically compatible gas in the form of extremely finely distributed small gas bubbles. A contrast medium is obtained which consists of small gas bubbles containing a gas and polyoxyethylene-660-12-hydroxystereate and a negatively charged anionic phospholipid in an aqueous solution.

This invention forms an ultrasound contrast medium on the basis of air or other, physiologically compatible, gases, which provides a clear contrast with the surrounding tissue, wherein the small gas bubbles are so small and stable that, following IV injection, they reach the left heart without qualitative, as well as quantitative losses. This is distinguished by good compatibility without any allergenic potential, wherein the small gas bubbles do not clump together either in the blood or in water, which is easily produced.

Advantageous further developments and embodiments in accordance with the invention can be taken from the features of the claims.

### DESCRIPTION OF PREFERRED EMBODIMENTS

The preparation to be described in what follows differs from the prior art because this invention is an echo contrast medium which, produced with little mechanical outlay, contains micro-air bubbles or micro-gas bubbles, and which is respirable because of its great stability.

For producing the IV contrast medium, this invention proposes a method and further developments of the method, for producing the IV contrast medium, as described in this specification and in the claims.

The contrast medium of this invention can be modified by varying the physiologically compatible gases to be incorporated. Thus, it is possible to respectively produce an echo contrast medium or NMR contrast medium of considerably longer shelf life from the same preparation by using gaseous fluorocarbons at body temperature, primarily C₃F₈ to C₆F₁₄. Using partially bromated fluorocarbons, a contrast medium is created which in accordance with this invention can be used as an X-ray contrast medium.

The gas (air) required for the contrast medium is added mechanically to the preparation. The volume of gas transported by the small gas bubbles is 0.01 to 0.1 ml per ml of preparation. The small gas bubbles are created by mechanical preparation and loaded with the gas in accordance with the respective indication.

The physiological isontonicity of the contrast medium can be produced by the addition of osmotically active substances.

It is possible by the method for producing the contrast medium of this invention to achieve size distributions of the small gas bubbles, 90% of which are smaller than 3 μm. 1 ml to 10 ml of the contrast medium are required per injection.

In accordance with this invention, the IV contrast medium is used as a foamed preparation for intravenous injection as a diagnostic agent in image-producing methods.

If the IV contrast medium in accordance with this invention is used as an ultrasound contrast medium, following the IV administration, not only the representation of the right ventricular portion of the blood circulation is possible.

Instead, the contrast medium can also be used with excellent results in examinations of the left heart and of the myocardium.

Other organs supplied with blood, such as the liver, the spleen, the kidneys and the brain, can also be made visible with the contrast medium of this invention.

But the contrast medium in accordance with this invention is also useful for making cavities in humans or animals visible, such as for the representation the bladder, the ureter, the uterus or the vagina.

After an IV administration, a solution consisting of polyoxyethylene-660-12-hydroxystereate and an anionic phospholipid is capable of clearly increasing an acoustical impedance in the arterial circulatory system by means of physiologically compatible gases incorporated by agitation.

This invention will be described in detail in the following two examples, wherein listed percentages always relate to the weight (g/v), unless otherwise indicated.

The aqueous solution contains 0.5 to 15 weight-% of polyoxyethylene-660-12-hydroxystereate (preferably 1 to 5 weight-%) and 0.1 to 10 weight-% of anionic phospholipids (preferably 0.5 to 2 weight-%). Physiologically suitable substances for setting the isotonicity can also be added to the solution.

### EXAMPLE 1

| | | |
|---|---|---|
| Polyoxyethylene-660-12-hydroxystereate | 3.00 | g |
| Dimyristoylphosphatidylglycerol | 1.00 | g |
| Sodium chloride | 0.90 | g |
| Distilled water | to make 100.00 | ml |

The product is manufactured in that the polyoxyethylene-660-12-hydroxystereate is melted at about 65 to 70° C. Thereafter, the anionic phospholipid is dissolved therein and then the water and the sodium chloride are slowly added while stirring. The pH value is set to 7.0 to 8.0 by means of diluted soda lye or hydrochloric acid.

### EXAMPLE 2

| | | |
|---|---|---|
| Polyoxyethylene-660-12-hydroxystereate | 2.00 | g |
| Dipalmitoylphosphatedidylglyceral | 1.00 | g |
| Glycin | 1.50 | g |
| Distilled water | to make 100.00 | ml |

The polyoxyethylene-660-12-hydroxystereate is melted at about 65 to 70° C. Thereafter, the anionic phospholipid is dissolved therein and then the water (65 to 70° C.) is slowly added. The glycin is dissolved. A pH of 7.0 to 8.0 is set by means of respectively diluted soda lye or hydrochloric acid.

All physiologically compatible gases can be used for the creation of gas microbubbles. 1 ml of the preparation in accordance with this invention is foamed up with 0.01 to 0.01 ml of gas. The microbubbles created in this way are preferably IV-injected. 1 to 20 ml of the preparation in accordance with this invention will be injected, based on the diagnostic formulation of the question.

Preparation of the Ready-To-Use Contrast Medium

If possible, the small gas bubbles should be created in the already known manner as shortly as possible before being administered to a patient. If the above-mentioned substance is offered in a pierceable boffle, for example, the solution can be drawn up, together with the required amount of respectively air or gas, into a customary commercial syringe, which is then connected with a second one in order to be able to pump the solution back and forth under pressure. The microbubbles are created by this method. The connecting piece with the second syringe can be a three-way cock, an adapter with a cross-sectional restriction or any other structure which has an effect on the fluid mechanics.

Echocardiographic Tests on Dogs

The previously drawn-up substance in accordance with this invention was pumped back and forth with the aid of two syringes, which are connected with each other by means of a three-way cock. The small gas bubbles created by this were injected into a peripheral vein. Rinsing with a physiological common salt solution was performed thereafter. A conventional ultrasound head suitable for echocardiography was placed in the location which is typical for the left and right heart prior to, during and after the injection.

Immediately following the injection, it was possible to trace on the monitor of the ultrasound device the way in which the blood marked by the contrast medium reaches the right ventricle through the right atrium and leaves it again by way of the pulmonary artery. Following passage through the lungs, the cavities of the left heart were very well visible, while those of the right heart emptied again. It was noted that the contrast medium of this invention remains a little longer in the left heart than in the right heart. The intensity of the contrast in the ultrasound image was almost identical in both halves of the heart, so that it must be assumed that the air contained in the small gas bubbles is not considerably reduced, so therefore a transport of the small gas bubbles almost free of losses is assured, even during the passage through the lungs to the left heart.

When repeating the test with small gas bubbles filled with gas (preferably with fluorocarbons), it was found that a length of contrast in both halves of the heart is increased.

The result of this was that, in contrast to the present technology in this field, the image-producing properties of the substance in accordance with this invention can be changed as required in a simple and reproducible manner by an exchange of the gases. By means of this the range of options in ultrasound diagnostics (heart, vessels, tumor perfusion, etc.) is considerably expanded.

The IV contrast medium in accordance with this invention is distinguished in that it can be diluted, infused, comparable with indications in radiology, used in connection with MRI, and/or recirculated.

Because of its great compatibility it is also possible to administer the IV contrast medium in accordance with this invention intra-arterially.

The polyoxyethylene-660-12-hydroxystereate employed in the examples has the following specification:

| | |
|---|---|
| Solidification point: | 25-30° C. |
| Saponification number: | 53-63 |
| Hydroxyl number: | 90-110 |
| Acid number: | ≦1 |
| Water content (acc. to Karl Fischer) | ≦0.5% |
| pH value, 10% in water | 6-7 |
| Color 20% in water | not darker than color solution G 5 |
| Viscosity, 30% in water (25° C.) | apprx. 12 mPa-s |
| Sulfated ash | 0.3% |
| Heavy metals | ≦10 ppm |
| Ethylene oxide | ≦1 ppm |

The determination methods can be found in currently valid DAB [German Pharmacopeia]/Ph. Eur., unless otherwise noted.

The composition consists of polyglykolester of 12-hydroxystearic acid (70%)=hydrophobic portion, polyethylene glykol (30%)=hydrophilic portion.

12-hydroxystearic acid (12 HSA) is the main fatty acid component, besides it, stearic acid and palmitic acid can also be identified.

We claim:

1. An IV contrast medium comprising a preparation of an aqueous solution of polyoxyethylene-660-12-hydroxystereate and an anionic phospholipid.

2. The IV contrast medium in accordance with claim 1, wherein the preparation on the basis of the aqueous solution of polyoxyethylene-660-12-hydroxystereate and the anionic phospholipid comprises a physiologically compatible gas in a form of extremely finely distributed small gas bubbles introduced by agitation.

3. The IV contrast medium in accordance with claim 2, wherein air is used as the physiologically compatible gas.

4. The IV contrast medium in accordance with claim 2, wherein gaseous fluorocarbons from a group from C₃F₈ to C₆F₁₄ are used as the physiologically compatible gas.

5. The IV contrast medium in accordance with claim 2, wherein gaseous, partially bromated fluorocarbons are used as the physiologically compatible gas.

6. The IV contrast medium in accordance with claim 5, wherein 90% of the small gas bubbles have an average diameter of less than 3 μm.

7. The IV contrast medium in accordance with claim 6, wherein an osmotically active substance selected from a sodium chloride and a glycin is contained for producing a physiological isotonicity.

8. The IV contrast medium in accordance with claim 7, wherein the aqueous solution contains 0.5 to 15 weight-% of the polyoxyethylene-660-12-hydroxystereate and 0.1 to 10 weight-% of the anionic phospholipid.

9. The IV contrast medium in accordance with claim 8, wherein a dimyristoylphosphatidyl-glycerol is the anionic phospholipid.

10. The IV contrast medium in accordance with claim 8, wherein a dipalmitoylphosphate-didylglycerol is the anionic phospholipid.

11. A method for producing an IV contrast medium for use as a diagnostic agent in connection with image-producing methods comprising: a preparation made by melting a polyoxyethylene-660-12-hydroxystereate at about 65 to 70° C. and dissolving an anionic phospholipid in a molten mass and then adding distilled water and one of a sodium chloride and a glycin while stirring.

12. The method in accordance with claim 11, wherein the distilled water is added in a state preheated to 65 to 75 ° C.

13. The method in accordance with claim 12, wherein a pH value of the preparation is adjusted to 7.0 to 8.0 by adding one of a soda lye and a hydrochloric acid.

14. The method in accordance with claim 13, wherein a physiologically compatible gas is added to the preparation at a volume ratio of preparation to gas of between 1 to 0.1 and 1 to 0.01 and small gas bubbles are created by agitation.

15. The method in accordance with claim 14, wherein at least 90% of the small gas bubbles created have an average diameter of less than 3 μm.

16. The method in accordance with claim 15, wherein a preparation of 0.5 to 15 parts by weight of the polyoxyethylene-660-12-hydroxystereate and 1 to 5 parts by weight of the phospholipid and 100 parts by weight of distilled water and 0.5 to 2 parts by weight of an osmotically active substance for adjusting a pH value is prepared and 100 parts by volume of the preparation are foamed up with 1 to 10 parts by volume of a physiologically compatible gas to form small gas bubbles.

17. Use of a foamed-up preparation for intravenous injection as a diagnostic agent in connection with image-producing methods, wherein the foamed-up preparation is made by drawing up the preparation according to claim 1 together with a physiologically compatible gas in a syringe, connecting the syringe with a second syringe and pumping the preparation and gas back and forth under pressure, thereby creating microbubbles within the preparation.

18. Use of a foamed-up preparation for intravenous injection as a diagnostic agent in connection with image-producing methods, wherein the foamed-up preparation is made by drawing up the preparation produced according to claim 11 together with a physiologically compatible gas in a syringe, connecting the syringe with a second syringe and pumping the preparation and gas back and forth under pressure, thereby creating microbubbles within the preparation.

19. The IV contrast medium in accordance with claim 1, wherein air is used as a physiologically compatible gas.

20. The IV contrast medium in accordance with claim 1, wherein gaseous fluorocarbons from a group from C₃F₈ to C₆F₁₄ are used as a physiologically compatible gas.

21. The IV contrast medium in accordance with claim 1 wherein gaseous, partially bromated fluorocarbons are used as a physiologically compatible gas.

22. The IV contrast medium in accordance with claim 1, wherein 90% of small gas bubbles have an average diameter of less than 3 μm.

23. The IV contrast medium in accordance with claim 1, wherein an osmotically active substance selected from a sodium chloride and a glycin is contained for producing a physiological isotonicity.

24. The IV contrast medium in accordance with claim 1, wherein the aqueous solution contains 0.5 to 15 weight-% of the polyoxyethylene-660-12-hydroxystereate and 0.1 to 10 weight-% of the anionic phospholipid.

25. The IV contrast medium in accordance with claim 1, wherein a dimyristoylphosphatidyl-glycerol is the anionic phospholipid.

26. The IV contrast medium in accordance with claim 1, wherein a dipalmitoylphosphate-didylglycerol is the anionic phospholipid.

27. The method in accordance with claim 11, wherein a pH value of the preparation is adjusted to 7.0 to 8.0 by adding one of a soda lye and a hydrochloric acid.

28. The method in accordance with claim 11, wherein a physiologically compatible gas is added to the preparation at a volume ratio of preparation to gas of between 1 to 0.1 and 1to 0.01 and small gas bubbles are created by agitation.

29. The method in accordance with claim 28, wherein at least 90% of the small gas bubbles created have an average diameter of less than 3 μm.

30. The method in accordance with claim 11, wherein a preparation of 0.5 to 15 parts by weight of the polyoxyethylene-660-12-hydroxystereate and 1 to 5 parts by weight of the phospholipid and 100 parts by weight of distilled water and 0.5 to 2 parts by weight of an osmotically active substance for adjusting a pH value is prepared and 100 parts by volume of the preparation are foamed up with 1 to 10 parts by volume of a physiologically compatible gas to form small gas bubbles.
